(19) United States
(12) Patent Application Publication
(10) Pub. No.: US 2024/0387019 A1
(43) Pub. Date: Nov. 21, 2024

(54) DIGITAL AND PERSONALIZED RISK MONITORING AND NUTRITION PLANNING SYSTEM FOR PRE-DIABETES

(71) Applicant: SOCIETE DES PRODUITS NESTLE S.A., Vevey (CH)

(72) Inventors: CHRISTIAN DARIMONT-NICOLAU, Lausanne (CH); VANESSA CAROLINE CAMPOS, Forel (CH); ERIC ANTOINE SCUCCIMARRA, Lausanne (CH); FLORENT DUDAN, St-Sulpice (CH); DANIELA PROZOROVSCAIA, Paudex (CH)

(21) Appl. No.: 18/693,741

(22) PCT Filed: Sep. 21, 2022
(86) PCT No.: PCT/EP2022/076163

§ 371 Date: Mar. 20, 2024

Related U.S. Application Data
(60) Provisional application No. 63/246,394, filed on Sep. 21, 2021.

(51) Int. Cl. G16H 20/60; G06F 16/906; G16H 50/30

(52) U.S. Cl. CPC G16H 20/60; G06F 16/906; G16H 50/30

(57) ABSTRACT

Methods and systems monitor risk and recommend dietary intake for prediabetes via a personalized digital platform. The methods can include generating an application for assessing a user-specific prediabetes risk, wherein the application prompts entry of user attributes to assess the user-specific prediabetes risk; receiving, from a user device, user attributes of a user to assess the user-specific prediabetes risk for the user; generating, based on a vectorization of the user attributes, a feature vector associated with the user; performing a clustering of a plurality of feature vectors comprising the feature vector associated with the user and a plurality of reference feature vectors; determining, based on the clustering, a prediabetes risk level of the user; and generating, based on the prediabetes risk level of the user and the user attributes, a recommendation for a dietary intake for the user.

### TECHNICAL FIELD

Certain aspects of the present disclosure generally relate to a digital personalized health monitoring system that provides recommended meal plans for pre-diabetes. Inputs for the system include, but are not limited to, nutrient intake, food preferences, gender, weight, and age of the proposed user.

### BACKGROUND

Prediabetes is a global health imperative affecting hundreds of millions of people worldwide. Prediabetes is a state in which blood sugar levels are higher than normal but not yet high enough to be diagnosed as diabetes. Doctors may sometimes refer to prediabetes as impaired glucose tolerance (IGT) or impaired fasting glucose (IFG). The World Health Organization (WHO) states that people with IGT or IFG may have a high risk of developing diabetes. Although there may be several ways to test if a person has prediabetes, the relative absence of clear symptoms of prediabetes means that individuals may not know that they have prediabetes.

Diabetes may be a result of the pancreas not making enough insulin, or cells of the body improperly responding to insulin. As there may be no known cure for diabetes, one way of preventing diabetes is by reversing prediabetes through lifestyle interventions. For example, diet recommendations that mitigate the onset of prediabetes may be helpful in preventing diabetes. There is a desire and need for a digital and personalized nutrition planning system that more effectively engages those diagnosed with or at risk of prediabetes, and provides practical and effective dietary intake recommendations. One way to assess the effect of a dietary intake on the health of a user is through the glycemic load of the dietary intake. A glycemic load (GL) refers to a number that estimates how much the dietary intake will raise a person's blood glucose level after consumption. Conventional methods of determining glycemic load overlook the nuances of food products, which may include a variety of glycemic-index lowering ingredients. There is a desire and need for digital and personalized nutrition monitoring and planning system based on a more accurate and reliable glycemic load measurement.

Various embodiments of the present disclosure address one or more of the shortcomings presented above.

### SUMMARY

The present disclosure presents new and innovative methods and systems for monitoring risk and recommending dietary intake for prediabetes via a personalized digital platform. In one embodiment, a method is provided that includes a computing device (e.g., an application server) generating (e.g., via an application programming interface), an application for assessing a user-specific prediabetes risk. The application may prompt entry of user attributes to assess the user-specific prediabetes risk. The computing device may receive, from a user device associated with a user, one or more user attributes of the user (e.g., to assess the user-specific prediabetes risk for the user). For example, the user attributes may include blood glucose levels, age, weight, gender, physiological data, food preferences, morbidities, etc. The computing device may generate, based on a vectorization of the one or more user attributes, a feature vector associated with the user. The feature vector associated with the user may be compared with (e.g., by plotting) reference feature vectors associated a plurality of other users or references. The computing device may perform a clustering (e.g., k-means cluster) of a plurality of feature vectors comprising the feature vector associated with the user and the plurality of reference feature vectors. The clustering may result in a discrete number of clusters representing different prediabetes risk levels. The computing device may identify or determine, based on the clustering, a prediabetes risk level of the user. Based on the prediabetes risk level of the user and the one or more user attributes, the computing device may generate a recommendation for a dietary intake for the user.

In some aspects, the computing device may send the user device a message, via an application, requesting that the user input physiological data (e.g., heart rate, blood pressure, blood glucose, etc.). The user device may provide the physiological data (e.g., via a wearable and/or accessory device that is communicatively associated with the user device), and this physiological data may be received by the computing device (e.g., as the one or more user attributes).

In some aspects, clustering may involve determining, based on a number of prediabetes risk levels, a number of clusters to be formed. The computing device may perform one or more iterations of: selecting, for each cluster of the number of clusters to be formed, a centroid feature vector among the plurality of feature vectors comprising the feature vector associated with the user and the plurality of reference feature vectors; and determining the average distance of each of the plurality of feature vectors. After the computing device determines a set of centroids that minimize the average distance between the centroids and the feature vectors, the computing device may terminate the iterations. The feature vectors belonging to each cluster of the number clusters may be identified. Each cluster may represent a prediabetes risk level associated with each cluster. The feature vectors of each cluster may thus be identified, e.g., to determine the users of each prediabetes risk level.

The computing device may determine a set of user-specific products for the user (e.g., based on the one or more user attributes and the prediabetes risk level of the user). The generated recommendation for the dietary intake may include one or more user specific products (e.g., a subset) from the set of user-specific products. In some aspects, the user may be prompted (e.g., via a message received via the application on the user device), to input one or more filters for user-specific products, including but not limited to, an activity level, a food sensitivity, a preferred diet, or a comorbidity. The computing device may filter, from the set of user-specific products, a user-specific product based on the one or more filters inputted by the user. In some aspects, the computing device may determine a threshold glycemic load recommended for users belonging to each prediabetes risk level. The computing device may filter, from the set of user-specific products, any user-specific products that satisfy the threshold glycemic load corresponding to the prediabetes risk level of the user. The filter set of user-specific products can thus be recommended as part of the recommended dietary intake.

In some aspects, the computing device may identify a periodic (e.g., daily, weekly, etc.) nutrient and/or calorie requirement for the user, and a meal nutrient and/or meal calorie requirement for the user. The recommended dietary intake make be compared to these requirements and may be refined and/or filtered further before being presented to the user.

In another embodiment, a system is disclosed for monitoring risk and recommending dietary intake for prediabetes via a personalized digital platform. The system may comprise one or more processors a memory. The memory stores instructions that, when executed by the one or more processors, cause the system to perform one or more methods described herein. In another embodiment, a non-transitory computer readable medium is disclosed for use on a computer system containing computer-executable programming instructions for monitoring risk and recommending dietary intake for prediabetes via a personalized digital platform. The instructions may comprise one or more steps, methods, or processes described herein.

The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE FIGURES

FIG. 1 illustrates a system for monitoring risk and recommending dietary intake for prediabetes via a personalized digital platform, according to an embodiment of the present disclosure.

FIG. 2 illustrates an example food products database according to an exemplary embodiment of the present disclosure.

FIG. 3 illustrates a flow diagram of an example method of monitoring risk and recommending dietary intake for prediabetes via a personalized digital platform, according to an exemplary embodiment of the present disclosure.

FIG. 4 illustrates a flow diagram of an example method of clustering users based on prediabetes risk levels, according to an exemplary embodiment of the present disclosure.

FIG. 5 illustrates a flow diagram of an example method of determining a glycemic index of a dietary intake option to monitor the risk of, and provide dietary intake recommendations for, prediabetes according to an exemplary embodiment of the present disclosure.

FIG. 6 includes tables showing example corrective factors for nutrients applicable in an example method of determining a glycemic index of a dietary intake option according to an exemplary embodiment of the present disclosure.

FIG. 7 illustrates a flow diagram of an example method of applying dietary intake recommendations for prediabetes according to an exemplary embodiment of the present disclosure.

FIG. 8 is a table showing an example daily requirement for nutrients, calories, and glycemic load, according to an example embodiment of the present disclosure.

FIG. 9 is a diagram illustrating the generation of meal plans based on prediabetes risk clusters according to an exemplary embodiment of the present disclosure.

### DETAILED DESCRIPTION OF EXAMPLE EMBODIMENTS

As discussed previously, prediabetes is a metabolic disorder characterized by elevated blood sugar levels that fall below the threshold to diagnose diabetes mellitus. Preventing or treating prediabetes may be an effective way to prevent diabetes mellitus and other cardiovascular diseases. A person's diet may be closely tied to the progression of prediabetes. For example, consumption of meal may typically lead to an increase in blood glucose levels, which may, under normal circumstances, cause the body to produce insulin. The insulin may cause the uptake of blood glucose from the bloodstream into certain cells of the body. The reduction of blood glucose may trigger the production of glucagon, which may result in activities that cause blood glucose levels to be restored (e.g., by triggering hunger and meal consumption).

The present disclosure relates generally to a personalized digital platform to increase nutritional awareness, monitor prediabetes risk, and recommend dietary intake to reverse prediabetes. The personalized digital platform may prompt the user to input relevant information to allow the platform to assess a risk category of the user. For example, the user may input his or her age, weight, gender, and daily dietary intake information. In some aspects, the user may be prompted to input physiological data (e.g., blood glucose information) via a biosensor or wearable device. The information received from the user (e.g., user attributes) may be organized as quantifiable data, e.g., via vectorization. The user attributes pertaining to the user may be compared to the user attributes associated with other users to determine prediabetes risk level of the user (e.g., through a clustering or other machine learning model). The personalized digital platform can determine dietary intake options that are suitable for each prediabetes risk level, e.g., based on the glycemic index (GI) or glycemic load (GL) associated with each dietary intake option. The dietary intake options may also be based on periodic (e.g., daily, weekly, etc.) nutrient requirements, in addition to meal nutrition requirements. Furthermore, the dietary intake options may be based on periodic (e.g., daily, weekly, etc.) calorie requirements, in addition to meal calorie requirements. In some aspects, dietary intake recommendations may be presented to the user as prediabetes friendly recipes and/or customized meal plans. As the user is prompted to incorporate the dietary intake recommendations into the user's lifestyle, the user may be monitored on a periodic basis (e.g. via prompting input of physiological data) for improvement.

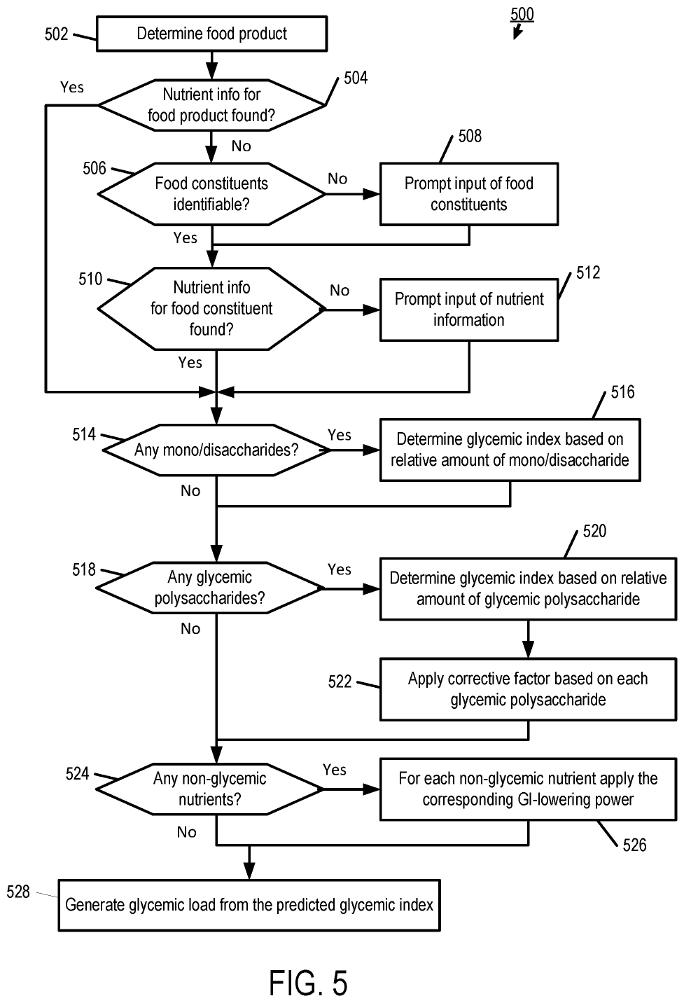

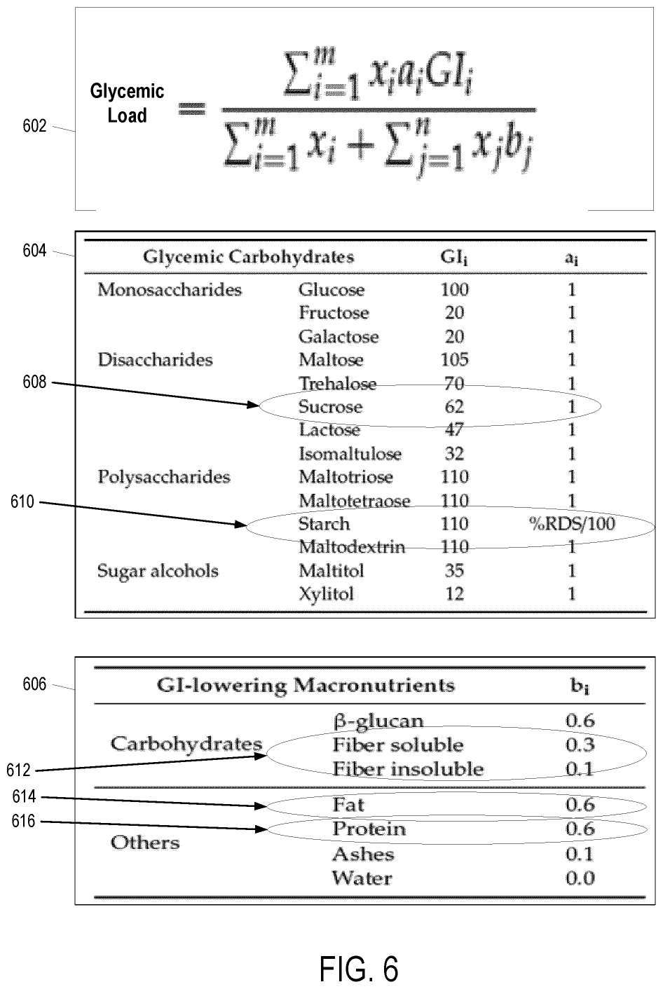

Additionally, the disclosure provides for a method for monitoring the risk of a user for prediabetes more reliably and accurately (e.g., via an improved method of determining a glycemic load for the diet of the user). A glycemic load refers to a number that estimates how much a given amount of food consumption (e.g., over a meal, over a day, over a prescribed length of time, etc.) will raise a person's blood glucose level after consumption. Conventional methods for determining a glycemic index and glycemic load involve aggregating the average GI and GL values of hundreds of common foods and beverages found in generic tables. However, such conventional methods do not reflect the specificities of the actual products (e.g., which depend on the local sources of ingredients and processes). Second, such conventional methods do not properly take into account the GI-lowering effects of the non-glycemic components in meals (e.g., the butter on the bread). Improved methods for determining GL and GI, and thus generating a more reliable and effective dietary intake recommendation for the user are discussed in more detail herein, for example, in relation to FIGS. 5 and 6. The disclosed digital and personalized nutrition monitoring system thus generates diet recommendations based on user-specific input and the improved assessment of glycemic load.

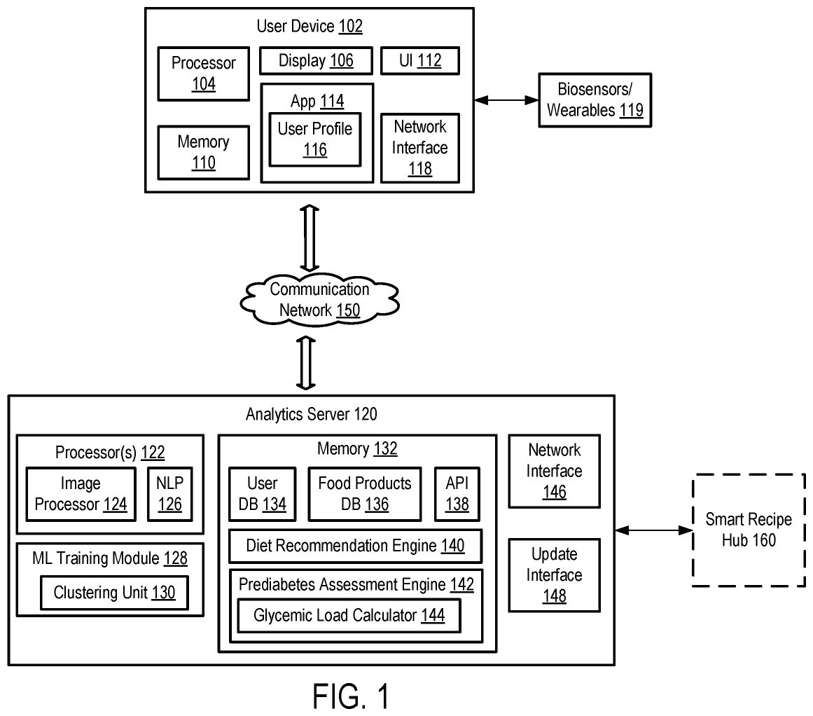

FIG. 1 illustrates a system 100 according to an embodiment of the present disclosure. The system 100 includes a user device 102 associated with a user and an analytics server 120 for performing one or more steps or methods described herein. The user device 102 may be communicatively coupled to one or more biosensors and/or wearable devices 119. Also or alternatively, the biosensors and/or wearables 119 may be a part of a standalone computing device (e.g., at a hospital and/or medical facility). Each of these devices and other external devices (not shown) may be able to communicate with one another over a communication network 150, which may be any wired or wireless network for disseminating information. Examples of the wireless networks may comprise Wi-Fi, a global system for mobile communications (GSM) network, and a general packet radio service (GPRS) network, an enhanced data GSM environment (EDGE) network, 802.5 communication networks, code division multiple access (CDMA) networks, Bluetooth networks or long term evolution (LTE) network, LTE-advanced (LTE-A) network or 5th generation (5G) network. Moreover, each device may include a respective network interface (e.g., network interface 118 and 146) to facilitate communication through the communication network 150. For example, the respective network interface may comprise a wired interface (e.g., electrical, RF (via coax), optical (via fiber)), a wireless interface, a, modem, etc. Furthermore, each of these devices may include one or more respective processor(s) (e.g., processors 104 and 122) and memory (e.g., memory 110 and 132) The processor may comprise any one or more types of digital circuit configured to perform operations on a data stream, including functions described in the present disclosure. The memory may comprise any type of long term, short term, volatile, nonvolatile, or other memory and is not to be limited to any particular type of memory or number of memories, or type of media upon which memory is stored. The memory may store instructions that, when executed by the processor, can cause the respective device to perform one or more methods discussed herein.

The user device 102 may be implemented as a computing device, such as a computer, smartphone, tablet, smartwatch, or other wearable through which an associated user can communicate with the analytics server 120, e.g., through use of an application 114. The user device 102 may further include a user interface (UI) 112, which may comprise a touch-sensitive display, a touchscreen, a keypad with a display device, or a combination thereof, and may work in relation to a display 106. The UI 112 may allow the first user to view audio, visual, and/or textual information presented by the analytics server 120 via application 114, access and use one or more applications 114, and enter input signals, e.g., by touching and moving icons on the display 106. The display 106 may comprise any medium of outputting visual information (e.g., image, video, etc.). The applications 114 may comprise any program or software to perform the methods described herein. For example, the applications 114 may include an application hosted by the analytics server 120 for monitoring nutrient levels and/or nutritional needs and recommending dietary intake. The user may have a user profile 116 associated with the application, and the user profile 116 may comprise a plurality of user attributes that may be utilized by the analytics server 120.

The user attributes may include biographical details about the user (e.g., a user identification, gender, weight, height, etc.), information about the health of the user (e.g., activity level, comorbidities, health goals, etc.), and dietary needs, dietary preferences and sensitivities, and allergies. Furthermore, the user attributes may include user entries to data structures (e.g., responses to standardized or unstandardized questionnaires) generated to assess prediabetes risk. In some embodiments, the user attributes may further include physiological data of the first user. The physiological data may be obtained via one or more biosensors and/or a wearable device that includes the one or more biosensors (e.g., “biosensors/wearables” 119). In some aspects, the biosensors/wearables 119 may be a part of, or may be communicatively linked to, the user device 102. The biosensors/wearables may include, but are not limited to, a glucose monitor, a sphygmomanometer, a heart rate measurement device, and/or devices that measure activity levels (e.g., FITBIT®).

The application 114 may comprise a personalized digital platform that recommends a diet plan (e.g., meal recommendations for an eating occasion or a period of time; guidelines based on nutrient, calories, and glycemic loads; etc.) based on the user's attributes and monitors the user's food intake and health.

The analytics server 120 may comprise a local or a remote computing system. The analytics server 120 may be configured for: requesting and receiving information received from the user device 102; processing information associated with a user or a follower; learning from various user-specific data concerning nutrient levels and user attributes to train machine learning models for predicting nutrient levels and nutritional needs; determining the glycemic load of various dietary intake options; generating a list of recommended products and uses of the products; and sending recommendations; among other functions.

The one or more processors 122 of the analytics server 120 may include an image processor 124 and a natural language processor 126. The image processor 124 may digitally process image data produced by the user device 102 to avoid noise and other artifacts, and prepare such image data for the recognition of texts and physical objects. The natural language processor 126 may be used to recognize texts, and determine meaning from texts captured from the image data. The texts may include the identifiers of user attributes and recognizable products (e.g., product name, company name, etc.). The image processor and/or natural language processor may rely on stored machine learning models from the machine learning module 128 to recognize, from image and text data, information for various user attributes (e.g., age and weight information from text in natural language, calories consumed per day from images of food, health conditions from text describing a health condition in natural language, etc.).

In one aspect, the machine learning module 128 may include a clustering unit 130. The clustering unit 130 may comprise a software, program, or instruction to identify clusters of feature vectors. The feature vectors may comprise values based on and corresponding to a set of user attributes for a plurality of individual users. For example, the values may correspond to user responses to requests for information to assess risks or severity of prediabetes. The clusters thus formed may indicate the risk level of a user. In some aspects, the clustering unit 130 may utilize an unsupervised machine learning model (e.g., Jenks natural breaks optimization, k means clustering, k-medians clustering, k-medoids clustering, fuzzy C-means clustering, Gaussian mixture models trained with expectation maximization algorithm, k-means++, etc.) to partition n observations (e.g., feature vectors) into k clusters, in which each observation belongs to the cluster with the nearest mean. In some aspects, the clustering unit 130 may utilize a supervised machine learning module (e.g., multiclass classification, multinomial logistic regression, k-nearest neighbors algorithm, etc.). In such aspects, the machine learning module 124 may also be used to train models to predict a specific class (e.g., prediabetes risk level) that a specific user may belong to, based on the user attributes associated with the user. The training may involve using reference training data. The reference training data may include feature vectors that each comprises values based on and corresponding to a set of user attributes for a plurality of individual users. The feature vectors may be associated with known prediabetes risk levels for those plurality of individual users, and may be trained as part of the supervised machine learning model.

The memory 132 of the analytics server 120 may further include a user database 134, a food products database 136, and an application program interface (API) 138. The user database 134 may store the respective user profiles associated with the users of the systems and methods presented herein (e.g., including the user associated with user device 102). The food products database 136 may store information about a plurality of recognizable (e.g., commercial) food products as well as food products for which information has been entered by various users of the personalized digital platform. The food products can comprise meals or items of a meal, and can have known nutritional information, calorie information, glycemic loads, and/or glycemic indices. The food products database 136 may list nutritional and calorie information for each product, uses of the product within recipes, and special instructions and warnings considering their usage. The food products may include food and beverage products associated with nutritional and dietary supplements. Example food products may include NUTREN DIABETIK, OPTIFAST, NUTREN MEALTIME COMPANION, or REDUCOSE.

The memory 132 may also include an application program interface (API) 138 that host, manage, or otherwise facilitate one or more applications in the user device 102 (e.g., application 114). For example, the API 138 may manage an application that enables the monitoring nutrient levels and recommending dietary intake. The diet recommendation engine 140, also referred to as the meal planner engine, may comprise one or more programs, applications, or implementations that generate appropriate recommendations for meals for a given user, including meals associated with a set of one or more products for the given user (e.g., user). The diet recommendation engine 140 may utilize the user database 134, food products database 136, and clustering unit 130. In some aspects, the diet recommendation engine 140 may utilize external databases, such as SMART RECIPE HUB 160, to provide dietary intake recommendations in the form of suggested recipes for meals.

The SMART RECIPE HUB 160 may comprise a repository of recipes for food products. Each recipe may be tagged by a meal or eating occasion (e.g., breakfast, lunch, dinner, snack, etc.) and a dish type (e.g., main dish, side dish, etc.). Furthermore, each recipe may provide nutritional information for the food product or a food product constituent associated with the recipe. In some aspects, SMART RECIPE HUB 160 may be a part of the analytics server 120.

The analytics server 120 may further include an update interface 148. The update interface 148 may comprise a database management program or application for managing one or more databases (e.g., user database 134, products database 136, etc.), such as via create, read, update, or delete (CRUD) functions. In some aspects, the update interface 148 may allow external devices (e.g., user device 102) to update one or more databases, e.g., such as when a new user would like to sign up to an application for monitoring nutrient levels and recommending dietary intake.

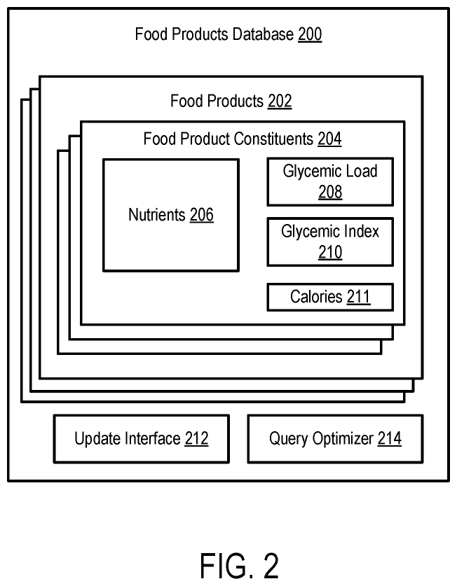

FIG. 2 illustrates an example food products database 200 according to exemplary embodiments of the present disclosure. As discussed previously, the food products database 200 may be a component of the analytics server 120. The food products database 200 may allow the analytics server 120 to identify a meal or food product that a user has consumed as part of monitoring the user or assessing a user's dietary habits to determine a prediabetes risk level for the user. Also or alternatively, the food products database 200 may allow the analytics server 120 to recommend dietary intake based on the prediabetes risk level of the user and user attributes associated with the user. The dietary intake recommendations for the user with respect to various food products may help the user to improve or overcome a prediabetic risk or condition. The food products database 200 may comprise profiles for a plurality of food products 202.

It is contemplated that some food products may comprise commercial or household food products whose food product constituents and ingredients may be known or easily accessible. However, profiles for other food products may be created by users and stored in the food products database 200, e.g., via update interface 212.

A food products profile 202 may include information about one or more food product constituents 204 (e.g., ingredients) that make up the food product. Furthermore, information for the nutrients 206 in each food product constituent 204 may be stored. The nutrients 206 information may include an amount or proportion of various glycemic carbohydrates, (e.g., monosaccharides, disaccharides, polysaccharides, sugar alcohols, etc.), GI lowering macronutrients (e.g., fibers, B-glucan, fat protein, ashes, water), vitamins, minerals, etc. Furthermore, for each food product constituent 204, the food products database 200 may store its glycemic load 208, glycemic index 210, and calories 211, any of the three may be based on the nutrients 206 in the food product constituent. In some aspects, the glycemic load 208 and glycemic index 210 may be automatically calculated based on the nutrients 206 of a food product constituent using methods presented herein (e.g., as will be described in relation to FIGS. 5 and 6). The glycemic load 208, glycemic index 210, calories 211, and/or nutrients 206 of individual food product constituents may be aggregated to determine glycemic loads, glycemic indices, calories and/or nutrients for a food product, a recipe, a meal, a daily food intake, a weekly food intake, etc.

The update interface 212 may comprise, be a part of, or perform functions similar to update interface 148, and may be used to update any aspect of the food products database 200. Furthermore a query optimizer 214 may allow external components and devices to more efficiently and accurately retrieve information from the food products database 200.

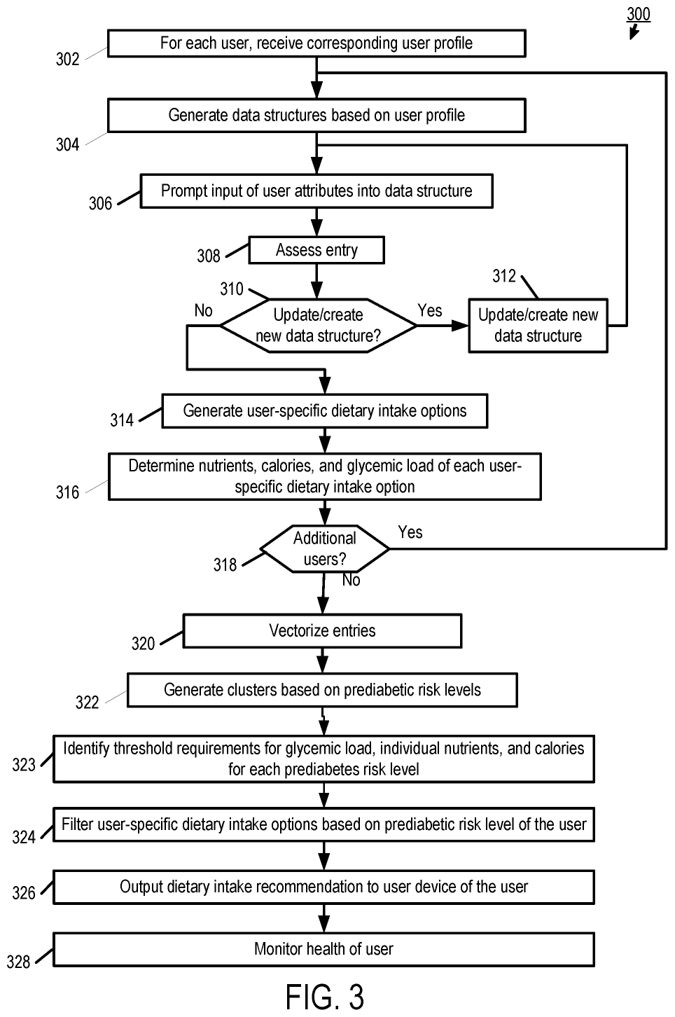

FIG. 3 illustrates a flow diagram of an example method 300 of monitoring risk and recommending dietary intake for prediabetes via a personalized digital platform, according to an exemplary embodiment of the present disclosure. Method 300 may be performed by one or more processors of the analytics server 120. Method 300 may concern the ability for a user of the application 114 to allow the analytics server 120 to determine a user's prediabetes risk level; determine nutritional, caloric, and glycemic load needs, recommend dietary intake, and monitor the user's prediabetes risk.

It is contemplated that a plurality of users may utilize or otherwise benefit from the personalized digital platform to improve their prediabetes risk or condition. Each user may be associated with a user device (e.g., user device 102) that the user may use to subscribe to the personalized digital platform (e.g., via application 114 that is managed by analytics server 120). Thus, referring to method 300, the analytics server may receive, for each user, a corresponding user profile (block 302). For example, as a condition for subscribing to the personalized digital platform (e.g., to receive the benefit of assessing and monitoring a prediabetic risk or condition of the user), the user may be prompted to submit, via the application 114 on the user device 102, preliminary information pertaining to the user such as identifying information of the user and/or user device (e.g., user name, user device number, age, weight, gender, etc.). The application 114 may be generated and hosted by API 134 of the analytics server 120, and the application may be accessible to users via their respective user devices. The application may be web browser enabled and/or may comprise a mobile application accessible on mobile platforms. Furthermore, the analytics server may track user devices that interact with the application (e.g., by storing device identifiers), and may recognize when the same devices come back to the application. The analytics server 120 may thus receive such preliminary information pertaining to the user at block 302. The preliminary information can be used to create a user profile for the user. The user profile may comprise a storage within user database 134 of analytics server 132 where additional information about the user (e.g., user attributed, prediabetes risk level, etc.) may be stored in data structures.

For example, for a given user, the analytics server may generate data structures based on the user profile (block 304). The analytics server may then prompt the input of user attributes into the data structure (block 306). As used herein, user attributes may include biographical details about the user (e.g., a user identification, gender, weight, height, etc.), information about the health of the user (e.g., activity level, comorbidities, health goals, etc.), dietary needs and preferences, and physiological data (e.g., blood glucose levels). In some aspects, prompting users to enter a user attribute may involve prompting the user to connect to a biosensor and/or wearable device (e.g., biosensors/wearables 119) to enter physiological data (e.g., blood glucose levels). Also or alternatively, an entry of a user attribute may be via an image, audio, and/or a video upload. For example, a patient data sheet may be scanned and uploaded on to the application 114, and the natural language processor 126 may parse and recognize user attributes pertaining to comorbidities, underlying health conditions, and biographical details. Preliminary information used for completion of the user profile may include one or more user attributes (e.g., age, weight, height, gender, etc.).

Furthermore, user attributes may be based on responses to standard questionnaires for assessment of risks and conditions for prediabetes or diabetes. For example, such questionnaires may include, but are not limited to the Finnish Diabetes Risk Score (FINDRISC), the Modified Asian FINDRISC (MODAsian FINDRISC), questionnaires provided by the American Diabetes Association (e.g., “Could You Have Diabetes? Take The Risk Test”), questionnaires offered by Diabetes UK (e.g., “Cheque For Tech”), etc.

It is contemplated that some input by users may not sufficiently respond to a prompt (e.g., question), or may engender a follow up prompt. Such situations serve as a reason for the analytics server to assess entries of the user via user device (block 308) and determine whether a data structure needs to be updated (e.g., for a supplemental or corrected response) or if a new data structure needs to be created (e.g., for a response to a follow up prompt) (block 310). If an entry does not sufficiently respond to a prompt or is a reason for a follow up prompt (e.g., question), the analytics server may update and/or create a new data structure for the user to correct or respond to a new prompt (block 312). The user may thus be again prompted to input user attributes into the updated or created data structure (block 306).

If responses to any prompts to input user attributes are deemed complete, the analytics server may generate user-specific dietary intake options (block 314). For example, the analytics server may prompt the user to enter, as user attributes, any dietary preferences or food allergies, comorbidities, preliminary information (e.g., age, weight, height, gender, etc.), or physiological data (e.g., blood glucose levels). Such user attributes may be used to determine daily nutrient and calorie requirements for the user (e.g., based on body mass index calculations), filter out any meals associated with the food allergies, and generate a list of food intake options based on the user's food preferences. The food options may include a list of known food products, recipes, and/or meals with known nutrition and glycemic index information. In some aspects, the user attributes may be used to determine requirements for individual nutrients and/or calories based on food group level rules. For example, certain food groups (e.g., vegetables) may have greater calorie requirements than other food groups (e.g., confectionaries). As will be described later, the generated user-specific dietary intake options may be further filtered based on the prediabetes risk level of the user, in subsequent steps.

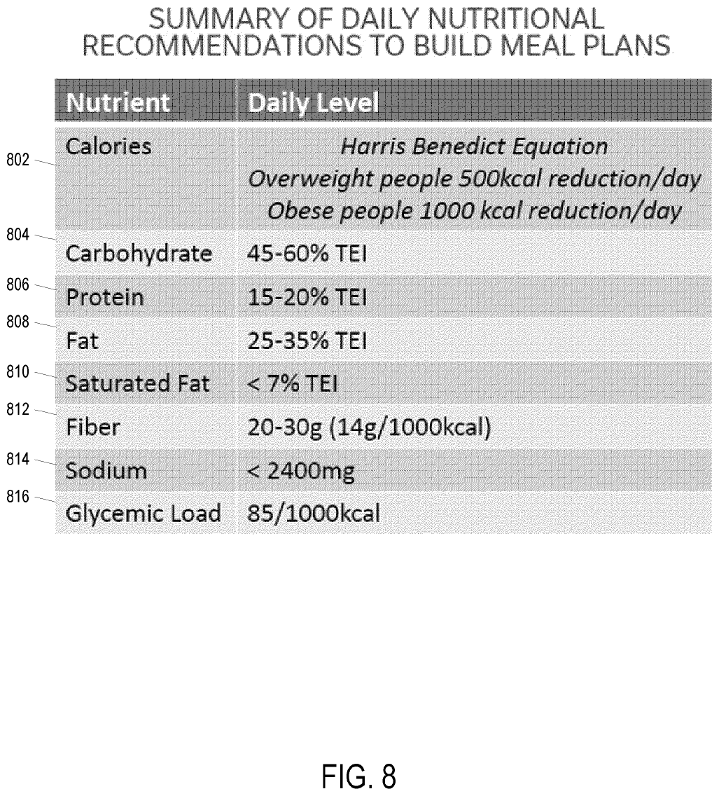

For example, the analytics server may determine the nutrients, calories, and glycemic load of each user-specific dietary intake option of the given user (block 316). As previously discussed, improved methods of determining the glycemic load of a given dietary intake option is disclosed, which leads to a more accurate and reliable monitoring of prediabetic risks and conditions. FIGS. 5 and 6 describe at least one embodiment of an improved method determining the glycemic load in more detail. The nutrients and calorie information may be retrieved, and/or otherwise calculated, from the food products database 136. As will be described further, in relation to FIG. 8, the nutrients may include but are not limited to carbohydrates, protein, fat (e.g., saturated fat, unsaturated fat, etc.), fiber, and sodium.

In order to classify the given user based on a prediabetes risk level, it may be helpful for the analytics server to have user attributes of other users. Thus, at block 318, the analytics server may determine whether the analytic server has identified any additional users for which user attributes can be received. If there are additional users, blocks 304 through 316 may be repeated for each of the additional users until a set of user attributes have been received, e.g., to determine prediabetic risk clusters in subsequent steps. If there are no additional users for which user attributes have not yet been received, or if there is sufficient data received from a plurality of users, the analytics server proceed to convert the inputted user attributes of the various users into quantifiable data (e.g., vectorize the user attributes of each user) (block 320). For example, a common set of m user attributes to which n users have inputted responses (e.g., age, weight, height, blood glucose level) may be arranged as n feature vectors of m dimensions. The individual responses may be converted to a numeric value within each feature vector.

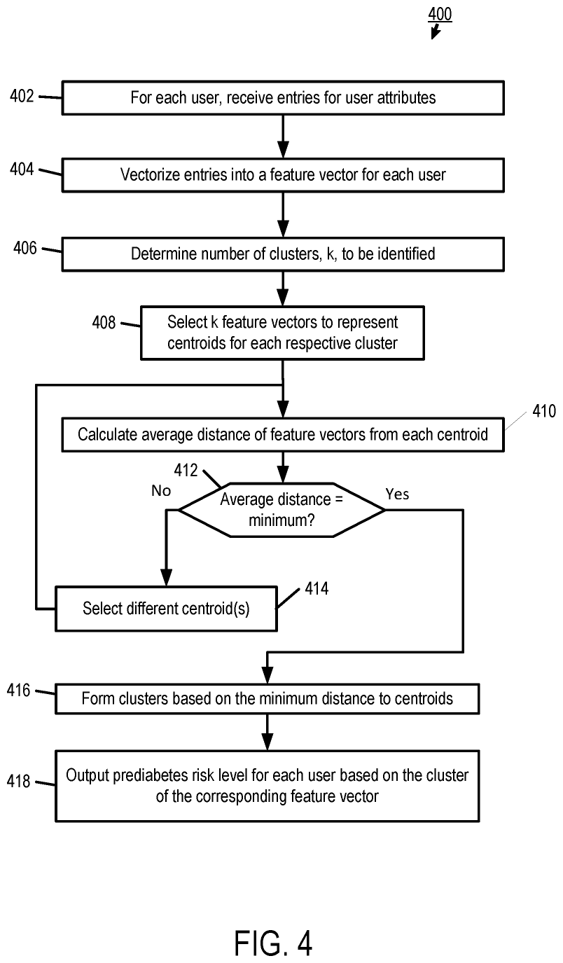

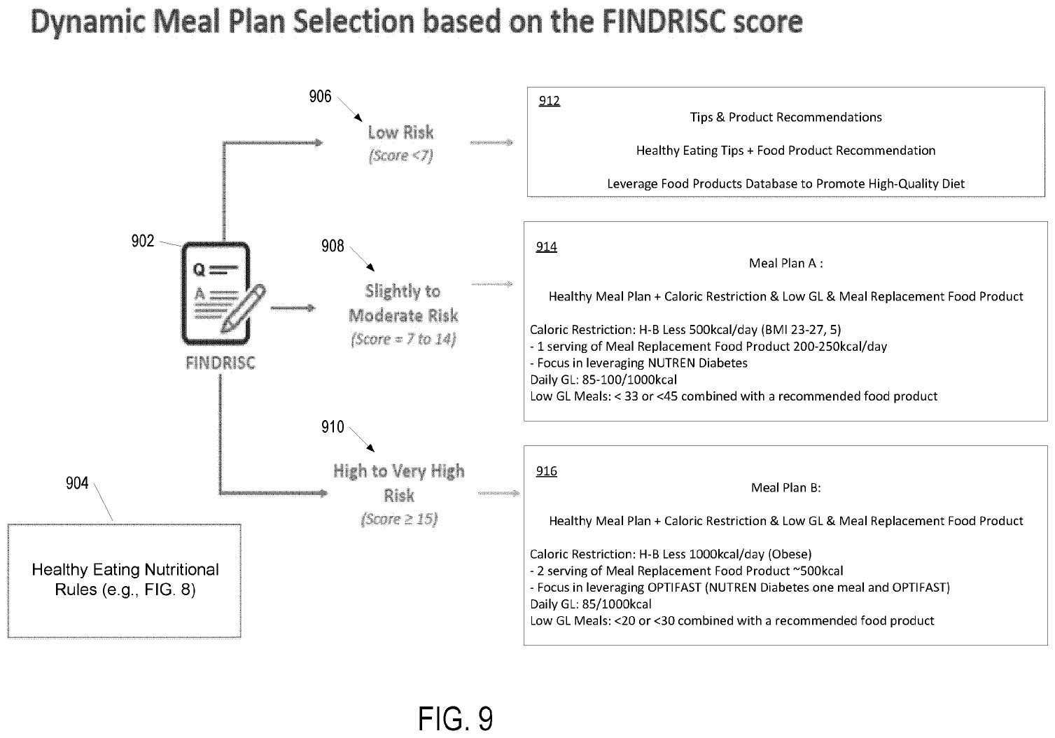

The analytics server may identify clusters from the feature vectors, the clusters signifying prediabetic risk levels (block 322). Unsupervised clustering procedures (e.g., k means clustering) or supervised machine learning models (e.g., multidimensional classification) can then be used to identify clusters among the feature vectors. FIG. 4 describes at least some embodiments of blocks 320 and 322 in more detail. The clusters thus formed and/or identified in block 322 may be used to classify each user according to a prediabetic risk level. In at least one embodiment, each cluster may correspond to a unique prediabetic risk level. The number of clusters to be formed may be predetermined, and the number of clusters may be varied depending on the level of specificity for dietary intake recommendation desired. For example, in one embodiment, one cluster may comprise users that are at a high to moderate risk for developing diabetes (e.g., Plan A), and another cluster may comprise users that are at a high to very high risk for developing diabetes (e.g., Plan B). As will be described further herein, FIG. 9 describes an example embodiment of three clusters (e.g., low risk, slightly to moderate risk, and high to very high risk), with example calorie restrictions, glycemic load requirements, and dietary intake recommendations for each cluster.

At block 323, the analytics server may identify threshold requirements for glycemic load, individual nutrients, and calories associated with each prediabetes risk level. The threshold glycemic load, nutrient, and calorie requirements associated with a given prediabetic risk level may be a recommended range for the glycemic load, nutrient, and calorie, respectively of a given meal for a user belonging to that given prediabetic risk level. In some aspects, each meal (e.g., breakfast, lunch, dinner, snack) may have a different threshold glycemic load, nutrient, and/or calorie requirement. As will be described further herein, FIG. 9 describes an example threshold requirements for glycemic load, nutrients, and calories associated with three different prediabetes risk levels.

At block 324, the analytics server may filter user-specific dietary intake options based on the prediabetic risk level of the user. For example, the analytics server may use the threshold glycemic load, nutrients, and calorie requirements associated with each prediabetic risk level. The analytics server may then identify, for each user, the user-specific dietary intake options (from block 314) and the glycemic load, nutrient, and calorie of each user-specific dietary intake option (from 316), in order to filter out those user-specific dietary intake options that do not satisfy the threshold glycemic load, nutrient, and/or calorie requirement associated with the user's prediabetic risk level. The resulting user-specific dietary intake options after the filtering may comprise the recommended dietary intake for the user.

At block 326, the analytics server may output the dietary intake recommendations for the user to the user device associated with the user. For example, the user may be able to view the recommendations on application 114 of the user device 102 for suggested meal options, e.g., for an upcoming meal (e.g., breakfast, lunch, dinner, etc.). In at least one embodiment, the analytics server may create a weekly (e.g., 7-day) meal plans with customized recipes, based on a user's food preferences and sensitivities. For example, recipes may be sourced from databases such as SMART RECIPE HUB, information regarding the individual food constituents of the recipes may be obtained from food products database 136, and then recipes may be filtered based on the prediabetes risk cluster and user attributes associated with the user. In some aspects, the analytics server may also include recommendations beyond dietary intake recommendations. For example, the health plan many include suggested exercises.

Furthermore, the analytics server may monitor the health of the user after (block 328). For example, the analytics server may prompt the user (e.g., by notifications sent via app 114) to input a description of the meal the user consumes (e.g., if not the dietary intake recommended by the analytics server). The analytics server may determine the glycemic load of the consumed meal and may then determine if it is within the targeted or threshold glycemic load associated with the prediabetes risk level of the user. Also or alternatively, the analytics server may periodically prompt the user to provide blood glucose levels via a biosensor or wearable device to track whether the recommended dietary intakes help improve the prediabetic condition of the user over time.

FIG. 4 illustrates a flow diagram of an example method 400 of clustering users based on prediabetes risk levels, according to an exemplary embodiment of the present disclosure. Method 400 may be performed by one or more processors of the analytics server 120. As previously discussed, method 400 shows at least one embodiment of blocks 320 and 322 of FIG. 3. Furthermore, as discussed in relation to blocks 306 of FIG. 3, the analytics server may prompt users of the personalized digital platform to enter responses for various questions that may be useful to assess prediabetes risk and recommend dietary intake options. These responses may be stored for each user as user attributes.

Thus, referring to FIG. 4, the analytics server may receive, for each user, the entries for user attributes (block 402). The entries may be stored in data structures associated with the user profile of each user. For purposes of determining clusters or classes of prediabetes risk levels, as discussed in FIG. 4, there may be a set of common user attributes (e.g., age, weight, height, blood glucose level, etc.) for which users may each have entries.

The analytics server may then vectorize the entries into a feature vector for each user (block 404). In some aspects, vectorization of entries may refer to converting the inputted entries for the user attributes of the various users into quantifiable data. A feature vector thus formed for each user may include the quantifiable data for each user attribute. For example, a common set of m user attributes to which n users have inputted responses (e.g., age, weight, height, blood glucose level) may be arranged as n feature vectors of m dimensions. The individual responses may be converted to a numeric value within each feature vector.

The analytics server may determine the number of clusters, k, to be identified (block 406). A cluster may refer to a group of data points (e.g., feature vectors) that naturally form as a group, e.g., due to their distances to one another when plotted in a multidimensional graph. Each cluster may signify a prediabetic risk level. The number of clusters to be identified may be based on how much stratification a user or operator of the personalized digital platform may desire in order to assess the prediabetes of users.

After deciding the number of clusters, k, to be identified, the analytics server may select k feature vectors that each represent a respective cluster (block 408). Such selected feature vectors may be referred to as centroids. Such selection may be arbitrary and/or randomized. For example, if the number of clusters, k, to be identified is 3, feature vector f₁ may represent cluster k₁, feature vector f₂ may represent cluster k₂, and feature vector f₃ may represent cluster k₃, even though the extent and composition of the clusters are still to be determined based on methods presented herein. It is contemplated the total number of feature vectors (e.g., data points) will be at least greater than k, such that each cluster to be identified may have at least more than one feature vector including the centroid. The analytics server may calculate the average distance of each feature vector from the centroids (e.g., the feature vector selected to represent each cluster) (block 410). The centroid from which the distance to a feature vector is calculated may be the centroid closest to the feature vector. The average may include a mean or a median of the distances of each feature vector from the respective centroid.

The calculated average distance may be assessed to determine if it is the minimum average distance (block 412). For example, the calculated average distance of one iteration of blocks 410 and 412 may be compared to a calculated average distance of previous iterations of blocks 410 and 412 to determine if a new minimum average distance is found. Until a minimum average distance is found, a different set of feature vectors may be selected as centroids to represent the k clusters to be identified (block 414). Thus, blocks 410, 412, and 414 may be repeated until a set of centroids are found that minimizes the average distance between each feature vector and their respective centroid. After that is found, the k clusters are identified based on the set of centroids that yield the minimum average distance (block 416). Thus, each centroid and its respective feature vectors that are closest to it may form a cluster. Furthermore, each cluster may represent a prediabetes risk level. For each cluster, the feature vectors that represent each user (e.g., via a vectorization of the user attributes of each user) may link the user to the prediabetes risk level associated with the cluster. The analytics server may thus output the prediabetes risk level for each user based on the cluster of the feature vector associated with the user (block 418). As discussed herein, the prediabetes risk level for the user may provide relevant information for recommending dietary intake for the user, e.g., by determining any threshold glycemic loads or other requirements associated with the prediabetes risk level.

FIG. 5 illustrates a flow diagram of an example method of determining a glycemic index of a dietary intake option to monitor the risk of, and recommend dietary intake for, prediabetes according to an exemplary embodiment of the present disclosure. Method 500 may be performed by one or more processors of the analytics server 120. Moreover, method 500 may describe at least some embodiments of block 316 of FIG. 3.

Method 500 may begin with identifying a food product (block 502). For example, the food product may comprise a dietary intake option for which the glycemic load is to be calculated (e.g., from block 316 of FIG. 3). Also or alternatively, the food product may be identified by a user of the personalized digital platform as being consumed or intended to be consumed by the user. For example, the user may enter an identification of or details regarding the food product via app 114 on user device 102.

The analytics server may determine whether nutrient information for the food product is available. For example, the analytics server 120 may query its food products database, 136 and 200, for the food product (e.g., food product 202) to see if there is any stored nutrient information 206 (e.g., based on any stored food constituents 204). If no nutrient is stored, the analytics server may determine whether there are any identifiable food constituents. For example, the analytics server may query its food products database, 136 and 200, to see if there are any food product constituents (e.g., ingredients of the food product) listed under the food product. If there are no identifiable food constituents (e.g., none are stored in the food products database, 136 and 200, the analytics server 120 may prompt the user to identify what an identified food product (e.g., peanut butter and jelly sandwich) is made of (block 508). The user may input the food constituents (e.g., two loaves of whole wheat bread, one teaspoon peanut butter, and one teaspoon strawberry jelly) via the app 114 on the user device 102. If information for food products constituents is found in the food product database 136, or after the user has provided information on the food product constituents, the analytics server may determine if any nutrient information for the food product constituent can be found in the food product database (block 510). For example, the analytics server 120 may query the food products database 136 and 200 for each individual food product constituent (e.g., whole wheat bread, peanut butter, strawberry jelly) to retrieve nutrient information associated with each food product constituent. In some aspect, each food product constituent may be stored under data structures associated with other food products. In such aspects, the analytics server 120 may update the data structure associated with the food product identified in block 502 to enter additional data fields for the food product constituents. Nutrients may include, but are not limited to, fats, proteins, water, vitamins, minerals, complex carbohydrates (e.g., glycemic polysaccharides), simple carbohydrates (monosaccharides, disaccharides, etc., and the like). If nutrient information is not found, the analytics server may prompt the user to input the nutrient information for the various food product constituents (block 512). The user may enter in nutritional information for each food product constituent via the app 114 of the user device 102. In some aspects, the nutrients may include, for example, carbohydryate, protein, fat, saturated fat, fiber, and sodium. In such aspects, recommended intake for such nutrients on a periodic (e.g., daily, weekly, etc.) basis may be provided to a user (e.g., based on a prediabetic risk level associated by the user).

Having retrieved (e.g., from its food products database) or received (e.g., from user device 102) nutrient information for the food product or the various food product constituents making up the food product, the analytics server may begin analysis of specific nutrients to determine the glycemic load of the food product. For example, the analytics server may determine if the food product has any monosaccharide or disaccharides (block 514). If there are, the analytics server may determine the glycemic index based on the relative amount of the monosaccharide or disaccharide (block 516). As will be discussed herein, FIG. 6 (e.g., via block 602) shows an example of how to determine the glycemic index in more detail.

Afterwards, or if there are no monosaccharides or disaccharides, the analytics server may determine if the food product has any glycemic polysaccharides (block 518). If there are, the analytics server may determine the glycemic index based on the relative amount of the glycemic polysaccharide (block 520). For example, the specific type of glycemic polysaccharide may be identified and the glycemic index may be retrieved from look up tables shown in block 604 of FIG. 6, and which may be stored in the food products database, 136 and 200. Furthermore, a corrective factor, aᵢ, based on each glycemic polysaccharide may be applied (block 522). As will be discussed herein, FIG. 6 (e.g., block 604) lists corrective factors, aᵢ, of various known glycemic carbohydrates.

Afterwards, or if there are no glycemic polysaccharides, the analytics server may determine if the food product has any non-glycemic nutrients (block 524). Such non-glycemic nutrients may include, but are not limited to, beta-glucan, fiber, fat, protein, ashes, and water. If there are, the analytics server may apply a GI-lowering power, bᵢ, associated with each non-glycemic nutrient in the determination of the glycemic index (block 526). For example, the specific type of non-glycemic nutrient may be identified from block 606 of FIG. 6, and the glycemic index may be calculated based on methods shown in block 602 of FIG. 6. As will be discussed herein, FIG. 6 (e.g., block 606) lists the GI-lowering powers, bᵢ, of various known non-glycemic nutrients.

Having identified the nutrients and the respective corrective factors, aᵢ, and GI-lowering powers, bᵢ, the analytics server may generate the glycemic load of the food product from the predicted glycemic index (block 528). For example, the analytics server may utilize the equation shown in block 602 of FIG. 6 to calculate the glycemic load.

FIG. 6 includes tables showing example corrective factors for nutrients to be applied in an example method of determining a glycemic index of a dietary intake option according to an exemplary embodiment of the present disclosure. For example, block 602 shows an exemplary method of determining a glycemic load based on the sum of the glycemic index GIᵢ of various nutrients xᵢ. As previously discussed, corrective factors aᵢ may be applied for nutrients such as glycemic carbohydrates. The sum may be divided by the sum of the nutrients plus the sum of any GI-lowering nutrients, xⱼ and their respective GI-lowering powers, bⱼ.

Block 604 is a table listing examples of various glycemic carbohydrates and simple carbohydrates. For example, glycemic polysaccharides include glycemic polysaccharides, and the simple carbohydrates include monosaccharides, disaccharides, and sugar alcohols. The glycemic index, GIᵢ, for these carbohydrates along with any corrective factors, aᵢ, are also listed.

Block 606 is a table listing examples of various GI-lowering nutrients. For example, such GI-lowering nutrients include carbohydrates such as Beta-glucan, fiber, fat, protein, ashes, and water. The GI-lowering powers, bᵢ, for the respective nutrients are also listed.

Furthermore, in at least one embodiment, methods for determining glycemic load and glycemic index may be simplified further. The simplified method may utilize five parameters-sucrose 608, starch 610, fiber 612, fat 614, and protein 616. The simplified method allows for a greater variety of food products to be more efficiently assessed for its glycemic index and/or glycemic load, as nutritional information for the five parameters may be more readily accessible. In some aspects, other nutrients may be linked to or otherwise placed within one or more of the five parameters based on their likeness or similarity in their corrective factor, aᵢ, or GI-lowering power, bᵢ.

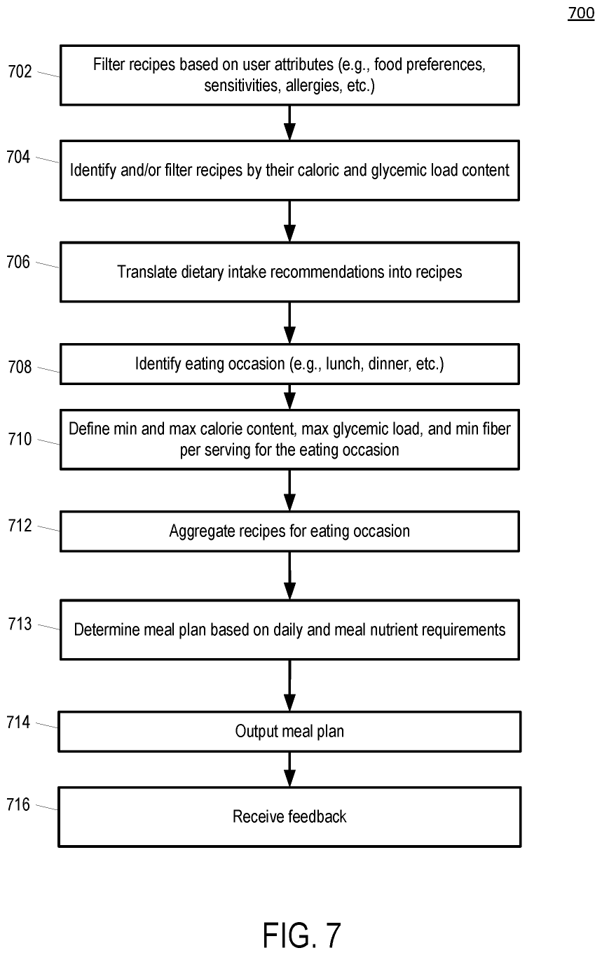

FIG. 7 illustrates a flow diagram of an example method 700 of applying dietary intake recommendations for prediabetes according to an exemplary embodiment of the present disclosure. Method 700 may be performed by one or more processors of the analytics server 120. Moreover, method 700 describe at least some embodiments of blocks 324 and 326 of FIG. 3, e.g., where dietary intake recommendations are provided in the form of meal plans based on recipes. Furthermore, method 700 may utilize an internal or external recipe database such as SMART RECIPE HUB 160, as previously described in relation to FIG. 1.

Referring to FIG. 7, method 700 may begin with filtering recipes in the recipe database (e.g., SMART RECIPE HUB 160) based on user attributes associated with the user (block 702). The user attributes may be those received by the analytics server 120 from the user device 102 associated with the user (e.g., at blocks 304 through 314), and may be used to perform a preliminary filtering of recipes from the recipe database. The user attributes may include, for example, food preferences and sensitivities (e.g., pork-free, beef-free, pescaterian, lactose-free, egg-free, etc.), and allergies (e.g., nuts, fish, shellfish, soy, gluten, etc.).

The recipes may be identified and/or filtered further by their caloric and glycemic load content (block 704). For example, each recipe may be associated with a food product and one or more food product constituents. If not already provided within the recipe database, method 500 previously discussed and shown in FIG. 5, may be used to identify and store some nutritional information (e.g., glycemic load and glycemic index) associated with the recipe. In some aspects, a preliminary filtering of the recipes based on caloric and glycemic load content may be performed (e.g., before a filtering based on a user's prediabetes risk level). For example, recipes with a caloric content per serving of more than 800 kcal and with a GL per serving of more than 20 may be filtered.

At block 706, the analytics server may translate any dietary intake recommendations into the recipes. For example, the analytics server has generated dietary intake recommendations based on a prediabetes risk level of a user, and that dietary intake recommendation prescribes a range for caloric intake and glycemic load, that range may be used to further filter the recipes.

Furthermore, for any specific dietary intake recommendation to be provided to the user, a meal or eating occasion (e.g., breakfast, lunch, dinner, snack, etc.) may be identified (block 708), e.g., for subsequent blocks 710 through 716. If a user has opted to receive dietary intake recommendations for meals over a span of time (e.g., a day, a week, etc.), the analytics server may perform subsequent steps for each of the meal or eating occasions constituting the span of time.

For each identified meal or eating occasion, the minimum and maximum calorie content, the maximum Glycemic Load and the minimum amount of fiber per serving may be defined (block 710). The prescribed calorie range for a complete meal or eating occasion may be more stricter than the calorie range allowable for an individual recipe as a meal may be based on several recipes of individual food products. Furthermore, the analytics server may choose to maximize the number of recipes used, e.g., by recommending a proportion (e.g., half) of a recipe in a meal whenever appropriate or as needed. Thus, for each identified meal or eating occasion, recipes of the individual food products constituting the meal may be aggregated together (e.g., in their appropriate proportions) (block 712).

In some aspects, a check may be performed to ensure that the aggregated combination fulfils daily requirements for individual nutrients and/or calories, and fulfils meal-level requirements for individual nutrients and/or calories. In some aspects, such requirements may be tied to a prediabetes risk level. Examples of daily and meal nutrient requirements are shown in FIGS. 8 and 9, described further herein.

The final meal plan may be outputted to the user device associated with the user (block 714). For example, the user may receive a week long (e.g., 7-day) meal plan with customized recipes for each meal, based on the user's food preferences and sensitivities. In some aspects, the analytics server may prompt the user (e.g., via app 114 of user device 102) to provide a feedback on a meal and/or meal plan, and may receive the feedback (block 716). The analytics server may use the feedback to edit and/or replace a subsequent meal in a meal plan, or change any food product constituents or their amounts.

FIG. 8 is a table showing an example daily requirement for nutrients, calories, and glycemic load, according to an example, non-limiting embodiment. In some aspects, the daily calories requirement 802 may be determined via the Harris Benedict equation (e.g., based on a basal metabolic rate (BMR). In the example shown, the daily calories requirement 802 may further show recommendations for the reduction of calories (e.g., overweight people—500 kcal reduction/day, obese people—1000 kcal reduction/day). As shown in the table, the individual nutrients and their daily intake requirements may include, for example, carbohydrates 804 (e.g., 45-60% TEI), protein 806 (e.g., 15-20% TEI), fat 808 (e.g., 25-35% TEI), saturated fat 810 (e.g., <7% TEI), fiber 812 (e.g., 20-30 g (e.g., 14 g/1000 kcal), a sodium 814 (e.g., <2400 mg). A daily requirement for glycemic load 816 is also shown (e.g., 85/1000 kcal). In some aspects, as dietary intake recommendations are generated, the analytics server may compare a dietary intake recommendation for individual meals of a day to ensure that the daily requirements are satisfied. The daily requirements may comprise a data structure stored in memory 132 of the analytics server 120, and may be updateable.

In some aspects, daily and mealtime requirements for calories, individual nutrients, and glycemic load (“nutritional rules”), such as those shown in FIG. 8, may be generated by synthesizing extracting information from nutritional guidelines of a plurality of organizations (e.g., The American Diabetes Association, Canadian Diabetes Association (e.g., including guidelines specifically for people with South Asian background), Diabetes UK, The Japanese Diabetic Society, National Diabetes Institute (Malaysia)). In some aspects, nutrition rules may be modified based on a target population. An example of nutritional rules thus formed from the extraction, filtering, and synthesis of guidelines is described in more detail in the Appendix. As shown in the Appendix, the nutritional rules include daily guidelines for macronutrients, caloric content, glycemic index and/or load, dietary patterns that may be diabetes friendly, fiber intake, carbohydrate intake, protein intake, fat intake, meal recommendations (e.g., including recommendations based on certain food groups (e.g., fruits, leafy vegetables, yogurt and cheese, etc.), supplement intake, salt intake (e.g., sodium), alcohol intake, sweeteners intake, and beverage intake.

In one embodiment, nutritional rules thus formed from a synthesis of these guidelines may include the following recommendations: a recommendation to eat at least 400 g of fruits and vegetable or above per day (e.g., excluding potatoes and other starchy root crops, but including legumes (e.g., beans, lentils), whole grains and nuts); a recommendation to limit intake of fats (e.g., as less than 30% of total calories), with a preference for the consumption of unsaturated fats to saturated fats, and a prohibition of trans fats; a recommendation to limit the intake of simple sugars to below 10% of total calories (e.g., as less than 25 grams or below 5% of calories per day); a recommendation to limit sodium and salt from all sources (e.g., below 5 g of salt per day), and to ensure that salt is iodized; and a recommendation to maintain healthy weight by consuming approximately the same number of calories the body is using.

FIG. 9 is a diagram illustrating the generation of meal plans based on prediabetes risk clusters according to an exemplary embodiment of the present disclosure. The prediabetes risk clusters may be formed based on user attributes obtained from users. Such user attributes may be obtained, for example, through responses to questionnaires for assessing prediabetes risk (e.g., as shown in block 902). The user attributes may be used to classify the user as belonging to one of a plurality of clusters (e.g., low risk 906, slightly to moderate risk 908, high to very high risk 910, etc.). In some aspects, the classification may be based on the generation of a score. The score may be based on a vectorization of the user attributes according to methods previously described. Tentative dietary intake recommendations may be generated based on each prediabetes risk cluster. Furthermore, such recommendations may be filtered based on food preferences, comorbidities, food allergies, and/or food restrictions. In addition, the tentative dietary intake recommendations may be further compared with nutritional rules governing a periodic (e.g. eating occasion, daily, weekly, etc.) food consumption. The nutritional rules may include guidelines for the threshold requirement for the intake of an individual nutrient, calories, or glycemic load, such as those shown in FIG. 8, as previously discussed. Based on a comparison with the nutritional rules, the tentative dietary intake recommendations may be filtered further.

For each user, a meal plan (e.g., dietary intake recommendation) may be generated based on the prediabetes risk cluster associated with the user. However, the meal plan may also be subject to, and/or further customized on the basis of, the nutritional rules 904, the user's food preferences, the user's comorbidities, the user's food allergies, and/or the user's food restrictions. For example, a user belonging to a low risk cluster 906 may receive tips and food product recommendations suitable for a low risk of prediabetes (e.g., block 912). For instance, the user may receive (e.g., via their app 114 on their user device 102, and from the API 138 of the analytics server 120), healthy eating tips and food product recommendations that may not be as restrictive as those for higher prediabetes risk clusters. The user may be promoted to leverage food products database 136 or a recipe repository (e.g., SMART RECIPE HUB 160) to promote a high-quality diet.

A user belonging to the slightly to moderate risk cluster 908 may receive a meal plan (e.g., Meal Plan A) suitable for that prediabetes risk level (e.g., as shown in block 914). The analytics server may recommend, to the user via their user device 102, a healthy meal plan, caloric restriction, a low glycemic load, and a meal replacement food product. The caloric restriction may involve reducing 500 kcal/day for users having a body mass index (BMI) of 23-27. The user may be recommended one serving of a meal replacement food product comprising, for example, 200-250 kcal/day. For example, the user may be prompted to leverage the meal replacement food product such as NUTREN DIABETES. The analytics server may recommend, to the user via user device 102, a daily glycemic load of 85-100/1000 kcal, and low glycemic load meal comprising a glycemic load that is less than 33, or less than 45 and combined with a recommended food product.

A user belonging to the high to very high risk cluster 910 may receive a meal plan (e.g., Meal Plan B) suitable for that prediabetes risk level (e.g., as shown in block 916). The analytics server may recommend to the user (e.g., via app 114 on their user device 102), a healthy meal plan, caloric restriction, a low glycemic load, and a meal replacement food product. The caloric restriction may involve reducing 1000 kcal/day for obese users. The user may be recommended two servings of a meal replacement food product comprising, for example, 500-kcal. For example, the user may be prompted to leverage meal replacement food products such as OPTIFAST and NUTREN DIABETES. The analytics server may recommend, to the user via user device 102, a daily glycemic load of 85 for 1000 kcal. Furthermore, the analytics server may recommend low glycemic load meal comprising a glycemic load that is less than 20, or less than 30 and combined with a recommended food product.

All of the disclosed methods and procedures described in this disclosure can be implemented using one or more computer programs or components. These components may be provided as a series of computer instructions on any conventional computer readable medium or machine-readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

It should be understood that various changes and modifications to the examples described here will be apparent to those skilled in the art. Such changes and modifications can be made without departing from the spirit and scope of the present subject matter and without diminishing its intended advantages. It is therefore intended that such changes and modifications be covered by the appended claims.

1. A method of monitoring risk and recommending dietary intake for prediabetes via a personalized digital platform, the method comprising:
generating, by a computing device having one or more processors and via an application programming interface, an application for assessing a user-specific prediabetes risk, wherein the application prompts entry of user attributes to assess the user-specific prediabetes risk;
receiving, by the computing device and from a user device associated with a user, one or more user attributes of the user to assess the user-specific prediabetes risk for the user;
generating, based on a vectorization of the one or more user attributes, a feature vector associated with the user;
performing a clustering of a plurality of feature vectors comprising the feature vector associated with the user and a plurality of reference feature vectors;
determining, based on the clustering, a prediabetes risk level of the user; and
generating, by the computing device and based on the prediabetes risk level of the user and the one or more user attributes, a recommendation for a dietary intake for the user.

2. The method of claim 1, wherein the performing the clustering comprises:
determining, based on a number of prediabetes risk levels, a number of clusters to be formed;
performing one or more iterations of:
selecting, for each cluster of the number of clusters to be formed, a centroid feature vector among the plurality of feature vectors comprising the feature vector associated with the user and the plurality of reference feature vectors; and
determining the average distance of each of the plurality of feature vectors;
identifying, based on a set of centroids that minimize the average distance, feature vectors belonging to each cluster of the number clusters; and
determining a prediabetes risk level associated with each cluster.

3. The method of claim 1, wherein the receiving the one or more user attributes comprises:
sending, to the user device via the application, a message requesting the user to input physiological data; and
receiving, from the user device, the physiological data.

4. The method of claim 1, further comprising:
determining, based on the prediabetes risk level of the user and the one or more user attributes, a set of user-specific products, wherein the generating the recommendation for the dietary intake comprises one or more user specific products from the set of user-specific products.

5. The method of claim 4, further comprising:
sending, to the user device via the application, a message requesting the user to input one or more of an activity level, a food sensitivity, a preferred diet, or a comorbidity; and
filtering, from the set of user-specific products, a user-specific product based on the one or more of the activity level, the food sensitivity, the preferred diet, or the comorbidity.

6. The method of claim 1, further comprising:
generating, by the computing device based on the one or more user attributes of the user, dietary intake options for the user; and
filtering, based on the prediabetes risk level of the user, the dietary intake options for the user to generate the recommendation for the dietary intake for the user.

7. The method of claim 6, wherein the filtering the dietary intake options comprises:
determining, for each dietary intake option, a glycemic load; and
comparing the glycemic load of each dietary intake option with a threshold glycemic load associated with the prediabetes risk level of the user.

8. A system for monitoring prediabetes risk and recommending dietary intake, the system comprising:
one or more processors; and
memory storing instructions that, when executed by the processors, cause the system to:
generate, via an application programming interface, an application for assessing a prediabetes risk, wherein the application prompts entry of user attributes to assess the prediabetes risk;
receive, from a user device associated with a user, one or more user attributes of the user to assess the prediabetes risk for the user;
generate, based on a vectorization of the one or more user attributes, a feature vector associated with the user;
perform a clustering of a plurality of feature vectors comprising the feature vector associated with the user and a plurality of reference feature vectors;
determine, based on the clustering, a prediabetes risk level of the user; and
generate, based on the prediabetes risk level of the user and the one or more user attributes, a recommendation for a dietary intake for the user.

9. The system of claim 8, wherein the instructions cause the processor to perform the clustering by:
determining, based on a number of prediabetes risk levels, a number of clusters to be formed;
performing one or more iterations of:
selecting, for each cluster of the number of clusters to be formed, a centroid feature vector among the plurality of feature vectors comprising the feature vector associated with the user and the plurality of reference feature vectors; and
determining the average distance of each of the plurality of feature vectors;
identifying, based on a set of centroids that minimize the average distance, feature vectors belonging to each cluster of the number clusters; and
determining a prediabetes risk level associated with each cluster.

10. The system of claim 8, wherein the instructions, when executed, cause the system to receive the one or more user attributes by:
sending, to the user device via the application, a message requesting the user to input physiological data; and
receiving, from the user device, the physiological data.

11. The system of claim 8, wherein the instructions, when executed, cause the system to:
determine, based on the prediabetes risk level of the user and the one or more user attributes, a set of user-specific products, wherein the generating the recommendation for the dietary intake comprises one or more user specific products from the set of user-specific products.

12. The system of claim 10, wherein the instructions, when executed, further cause the system to:
send, to the user device via the application, a message requesting the user to input one or more of an activity level, a food sensitivity, a preferred diet, or a comorbidity; and
filter, from the set of user-specific products, a user-specific product based on the one or more of the activity level, the food sensitivity, the preferred diet, or the comorbidity.

13. The system of claim 8, wherein the instructions, when executed, further cause the system to:
generate, based on the one or more user attributes of the user, dietary intake options for the user; and
filter, based on the prediabetes risk level of the user, the dietary intake options for the user to generate the recommendation for the dietary intake for the user.

14. The system of claim 13, wherein the instructions, when executed, cause the system to filter the dietary intake options by:
determining, for each dietary intake option, a glycemic load; and
comparing the glycemic load of each dietary intake option with a threshold glycemic load associated with the prediabetes risk level of the user.

15. A non-transitory computer readable medium for use on a computer system containing computer-executable programming instructions for monitoring prediabetes risk and recommending dietary intake, the instructions comprising:
generating, by the computing system and via an application programming interface, an application for assessing a user-specific prediabetes risk, wherein the application prompts entry of user attributes to assess the user-specific prediabetes risk;
for each of a plurality of user devices associated with respective plurality of users:
receiving, from the user device associated with the user, one or more user attributes of the user to assess the user-specific prediabetes risk for the user;
generating, based on a vectorization of the one or more user attributes, a feature vector associated with the user;
performing a clustering of a plurality of feature vectors associated with the respective plurality of users;
determining, based on the clustering, a prediabetes risk level of each of the plurality of users;
generating, for each of the plurality of users, based on the prediabetes risk level and the one or more user attributes of the user, a recommendation for a dietary intake for the user.

16. The non-transitory computer readable medium of claim 15, wherein the performing the clustering comprises:
determining, based on a number of prediabetes risk levels, a number of clusters to be formed;
performing one or more iterations of:
selecting, for each cluster of the number of clusters to be formed, a centroid feature vector among the plurality of feature vectors; and
determining the average distance of each of the plurality of feature vectors;
identifying, based on a set of centroids that minimize the average distance, feature vectors belonging to each cluster of the number clusters; and
determining a prediabetes risk level associated with each cluster.

17. The non-transitory computer readable medium of claim 15, wherein the instructions further comprise:
determining, for a given user of the plurality of users, based on the prediabetes risk level of and the one or more user attributes of the given user, a set of user-specific products, wherein the generating the recommendation for the dietary intake for the given user comprises one or more user specific products from the set of user-specific products.

18. The non-transitory computer readable medium of claim 17, wherein the instructions further comprise:
sending, to a given user device of the plurality of user devices via the application, a message requesting a given user associated with the given user device to input one or more of an activity level, a food sensitivity, a preferred diet, or a comorbidity; and
filtering, from the set of user-specific products, a user-specific product based on the one or more of the activity level, the food sensitivity, the preferred diet, or the comorbidity.

19. The non-transitory computer readable medium of claim 15, wherein the instructions further comprise:
generating, by the computing device and based on the one or more user attributes of a given user of the plurality of users, dietary intake options for the user; and
filtering, based on the prediabetes risk level of the given user, the dietary intake options for the given user to generate the recommendation for the dietary intake for the given user.

20. The non-transitory computer readable medium of claim 19, wherein the filtering the dietary intake options comprises:
determining, for each dietary intake option for the given user, a glycemic load; and
comparing the glycemic load of each dietary intake option with a threshold glycemic load associated with the prediabetes risk level of the given user.
